(19) United States
(12) Patent Application Publication
(10) Pub. No.: US 2013/0053751 A1
(43) Pub. Date: Feb. 28, 2013

(54) NEEDLE WITH AN ANTISEPTIC SWAB

(71) Applicant: Frank Holtham, Saddle River, NJ (US)

(72) Inventor: Frank Holtham, Saddle River, NJ (US)

(21) Appl. No.: 13/599,098

(22) Filed: Aug. 30, 2012

Related U.S. Application Data
(60) Provisional application No. 61/529,383, filed on Aug. 31, 2011.

(51) Int. Cl. A61M 35/00; A61M 5/32; A61M 5/34

(52) U.S. Cl. 604/1

(57) ABSTRACT

A disposable pen needle assembly for affixing to a pen injector for injecting a medicament, such as insulin, into a user. The pen needle assembly includes a needle that is sealed within a hub such that a proximal end of the needle is retained within the hub and enclosed by a peel-off hub inner seal. The opposite end of the needle extends outwardly for injecting the medicament. There is a sealed chamber external of the peel-off hub inner seal that contains a swab soaked with an antiseptic material. An outer removable seal, that also can be a peel-off seal, covers the sealed chamber containing the swab so that the user can simply remove the outer seal to gain access to the swab for cleansing the potential injection site. After swabbing the injection site, the hub seal is then also peeled off to access the needle for use in conventional manner.

### BACKGROUND

The present invention relates to a dispenser for medication having a disposable needle, and more particularly, to a multi-dosage medicament injector including a disposable needle assembly that is packaged to include an antiseptic swab for disinfecting the site of an injection.

There are, of course, presently existing, various types of syringes for administering some dosage of a medicament and many are intended for home use where the individual can self inject the medicament or have an acquaintance in the home carry out the administration of the injection.

Such injections are, for example, carried out daily or weekly in the case of a diabetic. As such, it is quite normal for such home syringes to be disposable, at least as far as the needle is concerned, so that the needle is used to administer the medicament and then disposed of appropriately, such as in a special sharps container.

One of the currently used common types of needles is the pen needle which is very popular with persons afflicted with diabetes. With a pen needle, there is a pen injector that includes a cartridge containing a multiple injection quantity of the medicament, insulin, and there are a number of disposable needles that are prepackaged along with the pen injector that can be sequentially affixed to a distal end of the pen injector to carry out the injection. As such, the pen injector contains a quantity of insulin capable of supplying multiple doses of insulin injections and the disposable needles are packaged as pen needle assemblies to be used with the pen needles.

Typically, the pen needle assemblies are packaged in an injected molded outer cover having a peel off foil seal and, after unpackaging the needle, the disposable needle is affixed to the distal end of the pen injector by a threaded engagement or other system to allow each individual pen needle to be affixed to the end of the pen barrel and then removed for disposal after the injection has been made.

It is important for the injection site where the needle is to be subcutaneously introduced into the user to be clean and free from germs on the skin prior to performing an injection. To that end, the user normally swabs the injection site with a swab of some antiseptic material. The swab may be pre-soaked with the antiseptic or may be used in conjunction with a bottle of an antiseptic and the swab soaked with the antiseptic just prior to the use of that swab.

Accordingly, one of the issues in the use of a home syringe is that it is not always convenient to have the swabs handy when it is time to carry out an injection and the user may need to look for the swabs. The swabs themselves are normally sold in large, bulky quantities of a large size, typically 2 inches by 2 inches, and at the time the user needs to make use of the syringe, the user's supply of swabs may be depleted or the user may be out of the antiseptic for soaking the swabs.

The lack of convenience in having swabs readily available to the user results in the user being frustrated in searching for available swabs and antiseptic material, to the extent that, at times, the frustrated user simply carries out the injection without the benefit of an antiseptic cleansing of the injection site and which is dangerous to the user and can result in an infection or worse.

It would therefore be advantageous to have a swab with the antiseptic presoaked therein and incorporated into the disposable pen needle assembly itself so that the user can have a fresh antiseptic swab available at the time when the needle is installed at the distal end of a pen injector so that the user has the convenience of always having an antiseptic swab handy when an injection is being conducted.

### SUMMARY OF THE INVENTION

Accordingly, the present invention relates to a pen needle assembly for attachment to a pen injector for providing injection of a medicament, such as insulin, to a user. The pen needle assembly includes a hub having a cylindrical shroud. The hub has a proximal end that is sealed by a removable seal, such as a peel-off seal, to create a closed chamber within the cylindrical shroud. There is a distal end of the hub that is closed and a needle passes through and is sealed to the hub such that the needle has a proximal end that is enclosed within the closed chamber within the cylindrical shroud and a distal end that extends outwardly from the closed end of the hub.

A needle protective cover surrounds and protects the distal end of the needle and a removable outer protective cover is affixed to the hub and encloses the distal end of the needle and the needle protective cover.

A closed chamber is formed that is external of the peel-off seal and which is also sealed by a removable outer seal, such as a peel-off seal, and a swab is located within the closed chamber that is soaked with an antiseptic material. As such when the user desires to use pen needle to carry out an injection of the medicament, the outer seal is removed so as to provide access to the swab. The user can then cleanse the injection site with the antiseptic soaked swab and, when the injection site has been sufficiently cleansed, the inner peel-off seal can be removed to provide access to the needle in the normal, conventional use of affixing the pen needle to a pen injector.

As such, with the present system, there is always an antiseptic soaked swab that is handy and available as a part of the overall pen needle assembly for the convenience of the user and to eliminate the need to hunt up a swab and source of antiseptic. When used, the swab can readily be disposed of and the pen needle installed in the conventional manner.

The present description will be referenced with respect to a pen needle, however, it will be seen that the present invention can be applicable to other types of medicament injectors and needle assemblies consistent with the spirit and intent of the present invention.

These and other features and advantages of the present invention will become more readily apparent during the following detailed description taken in conjunction with the drawings herein.

### BRIEF DESCRIPTION OF THE DRAWINGS

FIG. 1 is a perspective view of the conventional pen injector used to inject a medicament into a user;

FIG. 2 is an exploded view of a conventional pen needle assembly used with the present invention; and

FIG. 3 is a cross sectional view of a pen needle assembly constructed in accordance with the present invention.

### DETAILED DESCRIPTION OF THE INVENTION

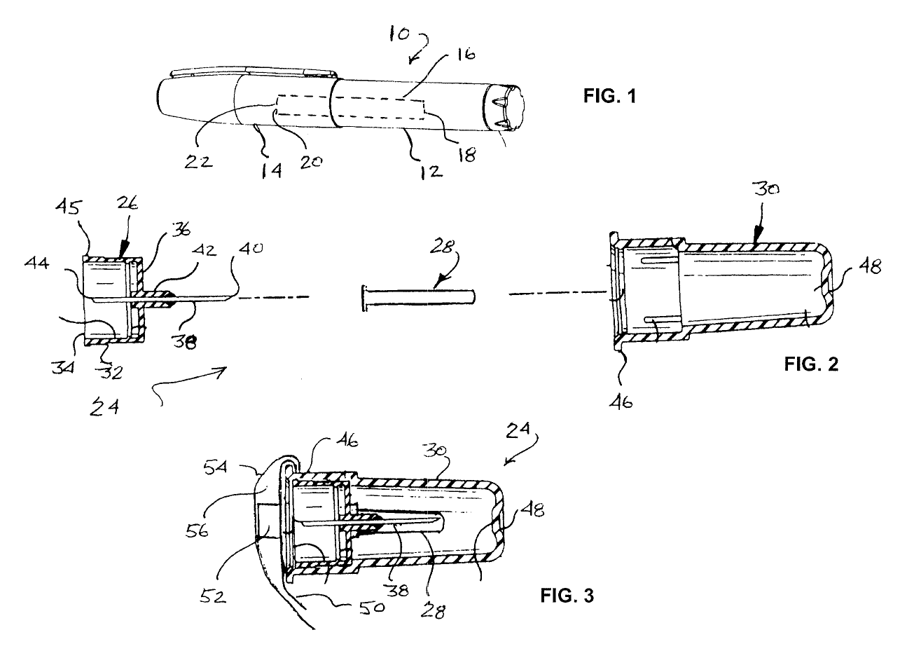

Referring now to FIG. 1, there is shown, a perspective view of a typical pen injector 10 that is usable with the present invention. As can be seen, the pen injector 10 includes main barrel 12 to which is affixed a removable cap 14 that covers the operative end of the pen injector 10.

Within the main barrel 12 there is a cartridge 16 containing a quantity of a medicament, such as insulin. The cartridge 16 has a closed end 18 and a septum end 20 that is covered by a septum 22 comprised of a material that can be easily pierced by a needle to access the medicament contained within the cartridge 16.

As such, in the operation of the pen injector 10, the cap 14 is removed by the user and a needle (not shown in FIG. 1) is used to pierce the septum 22, and provide access to withdraw the medicament from the cartridge 16.

Turning then to FIG. 2, taken along with FIG. 1, there is shown an exploded view of a conventional pen needle assembly 24 that is comprised of a pen needle 26, a needle protective cover 28 and a removable outer cover 30. As can be seen, the pen needle 26 is comprised of a needle hub 32 having an open proximal end 34 and a closed, distal end 36. The proximal end 34 is dimensioned to be affixed to the septum end 20 of the cartridge 16 during the operation of the pen injector 10 and that affixation may be by a threaded connection, interference fit or the like in a manner that is commercially available.

A needle 38 is sealed to the needle hub 32 and the needle 38 has a distal end 40 that extends outwardly from the needle hub 32 through a boss 42 and a proximal end 44 that is located within a cylindrical shroud 45 forming the proximal end 34 of the pen needle 26.

The needle protective cover 28 covers and protects the distal end 40 of the needle 38 and can be affixed to the needle hub 32 by a tight fit over the boss 42 so as to hold the needle protective cover 28 in place to protect the needle 38 and yet be easily removed by the user to gain access to the distal end 40 of the needle 38.

The outer cover 30 covers the needle 38 and the needle hub 32 and protects the needle hub 32 and the needle 38 when in place. Again, the outer cover 30 can have an open end 46 that is dimensioned to fit over the needle hub 32 to provide protection therefore. The outer cover 30 may be of a plastic material and has a distal, closed end 48.

As such, to summarize briefly, in the conventional use of a pen injector 10, the pen needle 26 is affixed to the pen injector 10 for use to inject the medicament into the user. The outer cover 30 is removed and the pen needle 26 is affixed to the septum end 20 of the cartridge 16 so that the proximal end 44 of the needle 38 pierces the septum 22 and is then in fluid contact with the medicament. The user can then remove the needle protective cover 28 and use the distal end 40 of the needle 38 to inject the medicament.

Turning finally to FIG. 3, there is shown a cross sectional view of a pen needle assembly 24 constructed in accordance with the present invention. As can be seen, there is a peel-off inner seal 50 that normally covers the open end 34 of the needle hub 32 to retain the cleanliness of the needle 38. As such, when the user is ready to use the pen injector 10, the user simply removes the peel off inner seal 50 to access the needle hub 32 so that the needle hub 32 can be attached to the pen injector 10.

In accordance with the present invention, there is a generally porous swab 52 that is located external of the peel-off inner seal 50 and can be affixed thereto. The porous swab 52 has been presoaked in an antiseptic liquid. The swab 52 is then covered by an outer seal 54, which, again, can be a peel-off seal, so that the swab 52 is retained in a sealed chamber 56 to prevent the evaporation of the antiseptic material and to retain that antiseptic material in sterile surroundings.

In use, therefore, the user initially removes the peel-off outer seal 54 which exposes the swab 52 so that the swab 52 can be used to rub against the injection site of the user to cleanse that area with the antiseptic. When the injection site has been sufficiently cleansed, the peel-off seal 50 can be peeled off to expose the proximal end 44 of the needle 38 so that the needle hub 32 can be affixed to the pen injector 10 in the conventional manner previously described.

The swab 52, outer seal 54 and peel-off seal 40 can be disposed of and the injection of the medicament carried out in the conventional manner. Once used, the needle hub 32, with the needle 38 affixed thereto, can be disposed in a sharps container or other safe location to dispose of the used needle.

Those skilled in the art will readily recognize numerous adaptations and modifications which can be made to the needle with an antiseptic swab of the present invention which will result in an improved needle assembly and method of using the inventive assembly, yet all of which will fall within the scope and spirit of the present invention as defined in the following claims Accordingly, the invention is to be limited only by the following claims and their equivalents.

In the claims:

1. A needle assembly comprising a hub, a needle sealed to the hub and having opposed ends, the hub having an open, proximal end enclosing one opposed end of the needle and having the other opposed end of the needle extending outwardly from the hub, an inner seal covering the open, proximal end of the hub, a swab containing an antiseptic material located external of the inner seal and a removable, outer seal enclosing the swab so as to enclose the swab in a closed chamber.

2. The needle assembly according to claim 1 wherein the needle assembly is a pen needle assembly.

3. The pen needle assembly of claim 2 wherein the inner seal is a peel-off seal.

4. The pen needle assembly of claim 2 wherein the outer seal is a peel-off seal.

5. The pen needle assembly of claim 1 wherein the swab is affixed to the inner seal.

6. The pen needle assembly of claim 2 wherein the pen needle assembly includes a needle protective cover that covers the opposed end of the needle extending outwardly from the hub.

7. The pen needle assembly of claim 2 wherein the assembly includes a removable outer protective cover

8. A needle injection system comprising an injector having a cartridge containing a medicament, a needle assembly comprised of a needle, a needle protective cover and a removable outer protective cover, the needle assembly having an outer chamber that is closed by a removable seal, the outer chamber containing a swab soaked with an antiseptic.

9. The needle injection system of claim 6 wherein the needle injection system is a pen needle injection system having a pen injector.

10. A method of using an antiseptic swab to cleanse an injection site of a user, the method comprising the steps of:
providing a needle injection system having an outer, sealed chamber containing a swab soaked with an antiseptic,
opening the outer, sealed chamber to expose the swab,
using the swab to cleanse an injection site of the user.

11. The method of claim 8 wherein the step or providing a needle injection system comprises a pen needle injection system.
